(19) United States
(12) Patent Application Publication
(10) Pub. No.: US 2011/0172750 A1
(43) Pub. Date: Jul. 14, 2011

(54) METHODS AND APPARATUS FOR ACTIVE PATIENT WARMING

(76) Inventors: David Ellsworth Cassidy, Chelmsford, MA (US); Bruce L. Carvalho, Watertown, MA (US)

(21) Appl. No.: 12/685,039

(22) Filed: Jan. 11, 2010

(51) Int. Cl. A61F 7/00

(52) U.S. Cl. 607/108; 607/96

(57) ABSTRACT

Methods and apparatus for warming a patient are disclosed herein. In some embodiments, a patient warming device may include a heater layer comprising a plurality of heater cells disposed on a flexible substrate; and a thermal conduction layer disposed on a patient side of the heater layer to transfer heat generated by the heater layer to a patient. The heater cells may comprise an electrical circuit to generate heat by Joule heating. The patient warming device is flexible to facilitate conforming the patient warming device to a patient during use.

### FIELD

Embodiments of the present invention generally relate to devices and methods for warming a patient.

### BACKGROUND

A body surface warmer is a device which helps maintain or increase a patient's temperature at or to a normothermic level by conducting heat into the body at the skin surface and utilizing the patient's circulatory system to distribute the heat to other portions of the body. Such devices can also be used in physical therapy or other areas requiring localized heating.

One common form of body surface warmer is a device in which warm air is pumped into an air mattress type blanket. Such blankets are typically large, and heat transfer can be poor since air is a poor carrier and conductor of heat. In some devices, warm water is pumped into a chambered mattress-type blanket. Water is a superior heat conveyer, but the water chambers are typically packaged in an insulator layer. In use, the insulator layer and air gaps limit thermal conductance to the patient.

Because there are large thermal resistances between the heated air or water medium and the patient, the medium is often heated to a temperature significantly higher than body temperature to obtain an adequate amount of heat transfer. The equipment to control such bladder-based blankets is often large and bulky. In addition, in the heated air versions, the equipment is noisy.

Other devices with resistive heaters have tended to have insulator layers, and have a propensity to form hot spots due to uneven generation of heat between or uneven conductance of heat away from portions of the resistive heater.

Thus, there is a need for improved methods and apparatus for surface warming a patient.

### SUMMARY

Methods and apparatus for warming a patient are disclosed herein. In some embodiments, a patient warming device may include a heater layer comprising a plurality of heater cells disposed on a flexible substrate; and a thermal conduction layer disposed on a patient side of the heater layer to transfer heat generated by the heater layer to a patient. The heater cells may comprise an electrical circuit to generate heat by Joule heating. In some embodiments, the resistive heaters may include a positive temperature coefficient (PTC) or a negative temperature coefficient (NTC) material having a switching temperature adapted to limit overheating as would be perceived by a patient. The use of miniature PTC resistive heaters or miniature NTC resistive heaters minimizes hot spots across the patient warming device because each heater can self-regulate independently. In some embodiments, a temperature sensor may be provided to provide closed-loop control of the power delivered to the heater cells and, therefore, the heat that is conveyed to the patient. In some embodiments, the electrical resistance across a heater cell may be measured during operation, thereby allowing the heater cell to be used as the temperature sensor. In some embodiments, a second temperature sensor may be placed in thermal contact with the heater cell, thereby facilitating accurate calibration of the heater cell as the temperature sensor. The patient warming device is flexible to facilitate conforming the patient warming device to a patient during use.

In some embodiments, a method of elevating the temperature of a patient or a portion of a patient may include applying the patient warming device as described in any of the embodiments herein to the patient; and generating heat with the patient warming device so that heat is conveyed to the patient.

In some embodiments, a patient warming device kit may include a patient warming device setup including a heater layer comprising a plurality of heater cells; and a thermal conduction layer adapted to be removably disposed on the patient warming device setup to form a patent warming device, wherein the thermal conduction layer is adapted to be disposed between the patient and the heater layer, wherein the patient warming device is adapted to be flexible to facilitate conforming to the patient.

In some embodiments, a replaceable thermal conduction layer may be provided so that the heater layer can be used with two or more patients. In some embodiments, a method of elevating the temperature of two or more patients or portions of the patients may include applying the patient warming device as described in any of the embodiments herein to a first patient; generating heat with the patient warming device so that heat is conveyed to the first patient; replacing the thermal conduction layer of the patient warming device; applying the patient warming device to a second patient; and generating heat with the patient warming device so that heat is conveyed to the second patient. Thus, portions of the patient warming device may be used at least twice before the device is disposed.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only illustrative embodiments of this invention and are therefore not to be considered limiting of its scope, for the invention may admit to other equally effective embodiments.

FIG. 1 schematically depicts use of a patient warming device on a patient in accordance with some embodiments of the present invention.

FIGS. 2A-B schematically depict perspective views of patient warming devices in accordance with some embodiments of the present invention.

FIGS. 3A-E illustrate top views of a heater cell for use in a patient warming device in accordance with some embodiments of the present invention.

FIG. 4 illustrates a top view of a plurality of heater cells for use in a patient warming device in accordance with some embodiments of the present invention.

FIG. 5 schematically depicts a perspective view of a patient warming device in accordance with some embodiments of the present invention.

To facilitate understanding, identical reference numerals have been used, where possible, to designate identical elements that are common to the figures. The figures are not drawn to scale and may be simplified for clarity. It is contemplated that elements and features of one embodiment may be beneficially incorporated in other embodiments without further recitation.

### DETAILED DESCRIPTION

Embodiments of the present invention include methods and apparatus for warming a patient. The inventive patient warming device generally provides local temperature control with few or no hotspots to enhance patient safety and to facilitate efficiently getting heat to the patient. The inventive patient warming device may be reusable or disposable. The inventive patient warming device operates without air or water flow, which facilitates simpler and quieter operation, with no air and/or water flow control mechanisms, and no fluid flows that can create contamination risk and/or maintenance issues.

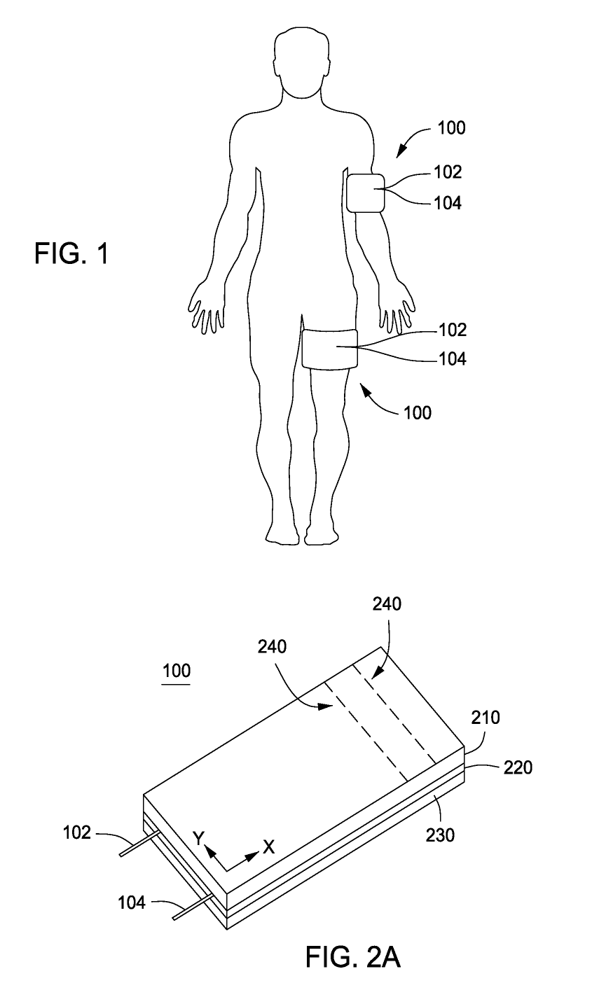

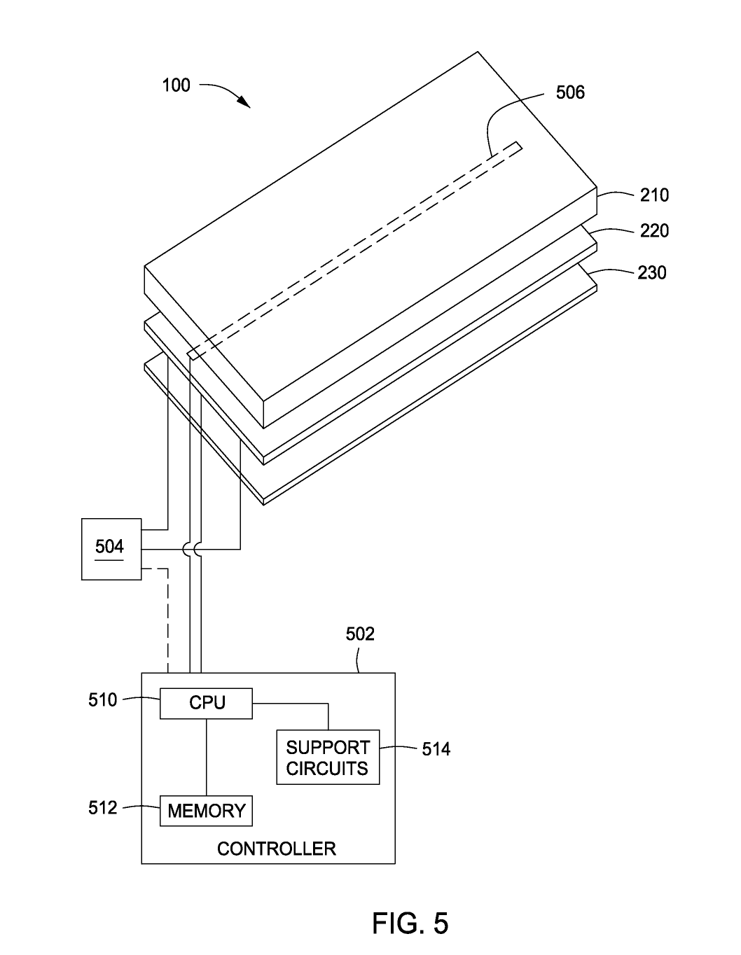

FIG. 1 illustratively depicts the application of two patient warming devices 100 to a patient. The patient warming devices 100 may be applied to the skin of the patient in any convenient location, for example, on the upper arm or thigh. The patient warming device 100 is a flexible device that can be wrapped around a patient, or a portion of a patient, such as about an arm or thigh as shown in FIG. 1. Pairs of leads 102, 104 may be coupled to a power source (shown in FIG. 5) to provide electrical current through the patient warming device 100. Generally, the patient warming device 100 may be applied to portions of body having higher blood circulation in order to enhance heat distribution within patient. It is thought that patient warming devices applied to the limbs can provide good thermal access to the human circulatory system and, therefore, efficient patient warming. Although FIG. 1 depicts two patient warming devices 100, greater or fewer patient warming devices (i.e., one or more) may be applied to a patient. In addition, it is contemplated that the patient warming devices may be placed on a patient in any location suitable for transferring heat to the patient from the patient warming device 100. Furthermore, the patient warming device 100 is removable, repositionable, and can be reusable or disposable, for more efficient use of the device. The patient warming device can thus be used to provide health care providers with flexibility in providing patient warmth while retaining needed access to other portions of the patient (for example, during surgical procedures).

The patient warming device 100 may generally have any desired size and geometry for a desired application. In some embodiments, the patient warming device 100 is sized so that it can be applied to the majority of human thighs without overlapping extra material. In some embodiments, the patient warming device 100 is sized so that it can be applied to the majority of upper arms (above the elbow) without extra, overlapping material. Because of the efficiency of heat transfer, it is believed that a relatively small-sized patient warming device, or multiple such devices, can provide the heat needed to convey sufficient heat (via the patient's circulatory system) in many low body temperature circumstances. The use of a small-sized device allows greater access to the patient than may be available with other heating devices. Moreover, the device's effectiveness despite small size allows it to be used in any of a number of body areas having good circulation that can be away from the area needing medical attention. This compact effectiveness allows a care giver to avoid using the patient's back—as is often done with other devices to warm while providing access. The back, while often out-of-the-way, has poor circulation, and the rib cage acts as a thermal insulator.

Additional non-limiting examples of areas that are useful for applying heat with the patient warming device 100 can include, the inner thigh, arms, side of chest, the gut, the neck, and the like, although any suitable location may be utilized. In some embodiments, the patient warming device 100 may be applied to a patient site with some pressure, such as provided by the patient's weight or with tape, ties, hook and loop fasteners, or other suitable binding agents or devices to maintain the desired position of the patient warming device. However, such pressure should not significantly hinder circulation. In most cases, it is believed that the patient warming device 100 can be applied for a significant period of time, such as 24 hours or more. Materials can be selected to minimize the number of patients susceptible to contact dermatitis due to the use of the patient warming device.

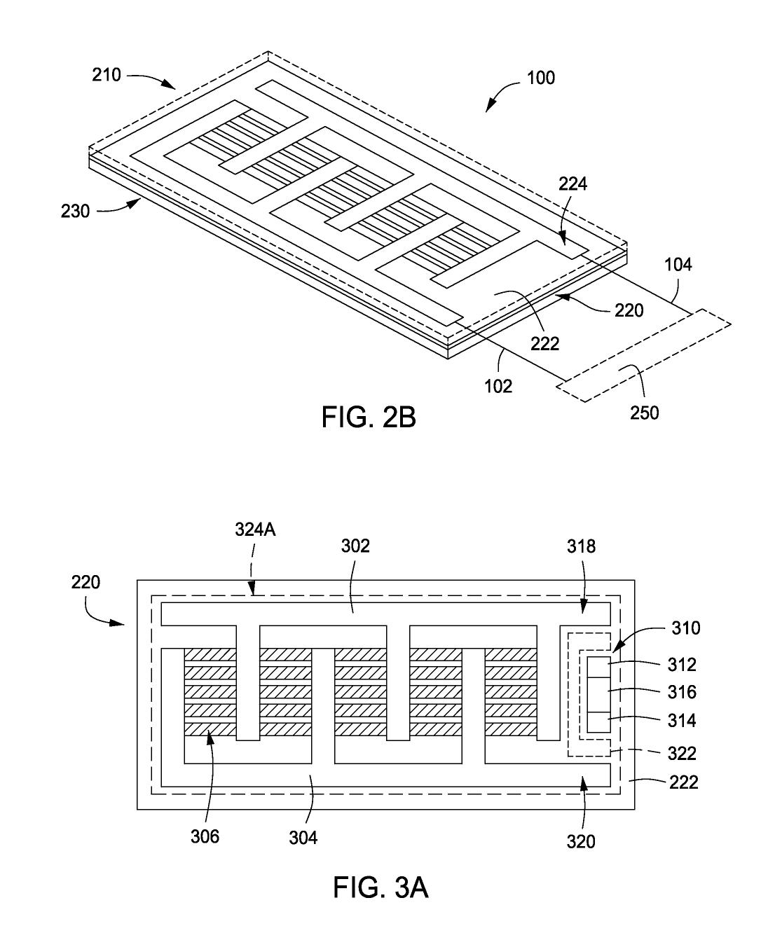

FIG. 2A is an oblique view of a section of the patient warming device 100 in accordance with some embodiments of the present invention. The patient warming device 100 generally includes a heater layer 220 for generating heat that is to be transferred to the patient and, optionally, either or both of a thermal insulator 210 disposed on a non-patient side of the heater layer and a thermal conduction layer 230 disposed on a patient side of the heater layer. In embodiments where both the thermal insulator 210 and the thermal conduction layer 230 are provided, they may be disposed on opposing sides of the heater layer 220, as shown in FIG. 2A.

In some embodiments, the thermal insulator 210 may be provided to restrict transfer of heat from the heater layer 220 in a direction away from the patient by insulating the patient warming device 100 from the environment (excluding the patient). Where heat output of the patient warming device is sufficient to provide the needed heat to the patient without the thermal insulator layer 210, such an insulator layer is not needed. In some embodiments, the thermal insulator 210 may be a thin, flexible closed-cell foam, although other flexible materials with a high internal surface area and low thermal conduction are expected to provide sufficient thermal insulation. For example, in some embodiments, the thermal insulator 210 may have a thermal conductivity that is within about 10 to about 20 percent of the average thermal conductivity of a human body. In certain applications, such as in a hospital or other setting with sufficient power provided, for example, via a wall outlet, the thermal insulator may not be necessary. In certain applications, such as in a field setting or other situation where relatively low power is provided, for example via a battery, a thermal insulator may facilitate more efficient use of available power by minimizing heat loss to the environment. In some embodiments, the thermal insulator 210 may be a layer disposed adjacent the heater layer 220 on a side opposite a patient side of the heater layer 220. In some embodiments, the thermal insulator 210 may be coupled, permanently or removably, to the heater layer 220, for example by gluing (for example, with adhesives or the like), heat diffusion, solvent bonding, welding, ultrasonic welding, mechanical fasteners (such as tying, hook and loop fasteners, taping, or the like), or other suitable fastening method. In some embodiments, the thermal insulator 210 may be placed near the heater layer 220 without coupling the thermal insulator 210 to the heater layer 220 (for example, merely placed atop the heater layer 220).

In some embodiments, a thermal conduction layer 230 may be provided to enhance thermal conduction between the heater layer 220 and the patient. The thermal conduction layer 230 may be thermally coupled to the heater layer 220 in any suitable manner that facilitates robust and uniform heat transfer from the heater layer 220 to the thermal conduction layer 230. As used herein, thermally coupled means coupled in a manner that minimizes or eliminates air pockets or other thermally insulative materials between the conduction layer 230 and the heater layer 220. The minimization or elimination of such air pockets or thermally insulative materials facilitates robust and uniform heat transfer from the heater layer 220 to the thermal conduction layer 230. As used herein, “thermal conduction” shall mean the ability to conduct heat energy from one location to another with minimal temperature drop. In some embodiments, the thermal conduction layer 230 may be disposed immediately adjacent to the heater layer 220. The thermal conduction layer 230 may be adhered to the heater layer by used of adhesives, welding, sonic welding, or by a natural affinity to bond between the materials of the heater layer 220 and the thermal conduction layer 230. In some embodiments, the dielectric substrate 222 of the heater layer may be treated, such as by used of a primer, a plasma treatment (such as corona treatment), or the like, to enhance the bond between the heater layer 220 and the thermal conduction layer 230.

The thermal conduction layer 230 is disposed between the patient's skin and the heater layer 220 and typically comprises a material and is of a thickness that promotes good skin contact with the patient. The thermal conduction layer 230 may also provide a volume through which the heat generated by the heater layer 220 may diffuse to become more uniform and further avoid local hot spots. The thickness of the thermal conduction layer 230 may be selected to balance the diffusion of the heat with efficiency of the device. In general, a thicker thermal conduction layer 230 will provide greater diffusion across the plane of thermal conduction layer (i.e., the X-Y plane shown in FIG. 2A). The configuration of the heater layer 220 may also be controlled to provide an acceptable thermal profile of the patient warming device 100. Thus, the configuration of the heater layer 220 and the thermal conduction layer 230 may be selected to minimize hot spots while retaining efficient operation of the patient warming device 100. In some embodiments, the thickness of the thermal conduction layer 230 is at least about 0.5 mm. In some embodiments, the thickness of the thermal conduction layer 230 is less than about 3 mm. In some embodiments, the thickness of the thermal conduction layer 230 is between about 0.75 and about 1.5 mm.

The thermal conduction layer 230 may comprise any suitable thermally conductive material that may be used in a health care setting. In some embodiments, the thermal conduction layer 230 must be sterilized, or be sterilizable. In some embodiments, where sterilization is not an issue, the thermal conduction layer 230 need not be sterilized (or sterilizable).

In some embodiments, the thermal conduction layer 230 may comprise a gel selected to have good thermal conductivity and good flexibility. In certain embodiments, the gel is sufficiently moldable so that it can be conformed to the shape of the patient to a further extent than would be provided by the flexibility of the patient warming device 100. In some embodiments, the gel is more thermally conductive, and in some embodiments significantly more conductive, than the patient's fat layer, such as a human dorsal fat layer. In some embodiments, the gel can wet the patient's skin, including conforming to the pores of the patient's skin, to reduce or eliminate a source of thermal impedance cased by air trapped between the patient warming device and the patent.

The gel can be, for example, a hydrogel, formed of natural or synthetic polymers. Hydrogels typically contain a high water content, and are thereby generally thermally conductive. Hydrogels are often used in wound care, and accordingly, it is believed that the gel material can be selected so that adequate skin compatibility should be obtained. Gels, such as hydrogels, can be formed, for example, of polyacrylamide copolymer, ethylene maleic anhydride copolymer, cross-linked carboxy-methyl-cellulose, polyvinyl alcohol copolymers, cross-linked polyethylene oxide, starch grafted copolymer of polyacrylonitrile, hydrocolloid materials (such as sodium or calcium carboxymethylcellulose, pectin, gelatin, guar gum, locust bean gum, collagen, gum karaya, and the like) and the like. The gels can be dispersed in a foam structure, thus comprising gel filled foams. One example of a hydrogel suitable for use in a patient warming device as described herein is commercially available from Katecho Inc., located in Des Moines, Iowa.

In some embodiments, the gel may be temporarily enclosed in, or covered with a plastic film, for example, to keep the gel from dehydrating. The covering film may be completely or partially removable and may be removed, for example, prior to applying the patient warming device to a patient in order to maximize thermal contact between the thermal conduction layer 230 and the patient. Films used to optionally enclose or cover the gel can be, for example, polyester (such as polyethylene terephthalate, polyethylene naphthalate, or the like), polyethylene, or the like, including any film that protects the gel layer and keeps it from dehydrating.

In some embodiments, the thermal conduction layer 230 may be removably coupled to the heater layer 220 to facilitate re-use of the remainder of the patient warming device. For example, after an initial use on a first patient, the thermal conduction layer (which was in contact with the first patient) could be removed and a new thermal conduction layer 230 could be provided and coupled to the heater layer 220 to facilitate providing a clean surface for applying the patient warming device to a second patient. In some embodiments where the thermal conduction layer 230 is a gel material, removal of the patient warming device may damage the thermal conduction layer 230. As such, removing the remainder of the thermal conduction layer 230 gel material and applying a new thermal conduction layer 230 may facilitate reuse of the patient warming device. Alternatively it is contemplated that the patient warming device may be a disposable, single-use device.

In some embodiments, components of the patient warming device may be packaged assembled and ready for use. In some embodiments, components of the patient warming device may be packaged separately as a kit. For example, a foil, polymer (such as Tyvek®), or other suitable material package may be provided with the heater layer 220 and optionally, thermal conduction layer 230 and/or thermal insulator 210, separately disposed therein. Upon opening the pack, the components of the patient warming device may be assembled for use. In some embodiments, the heater layer 220 and the thermal insulator 210 may be coupled together with only the thermal conduction layer 230 being separate. In some embodiments, the thermal conduction layer 230 may be provided in a package separate from the other components of the patient warming device so that a new thermal conduction layer 230 may be provided when the patient warming device is being re-used. In some embodiments, the package, and the components packaged therein, may be sterilized or sterilizable.

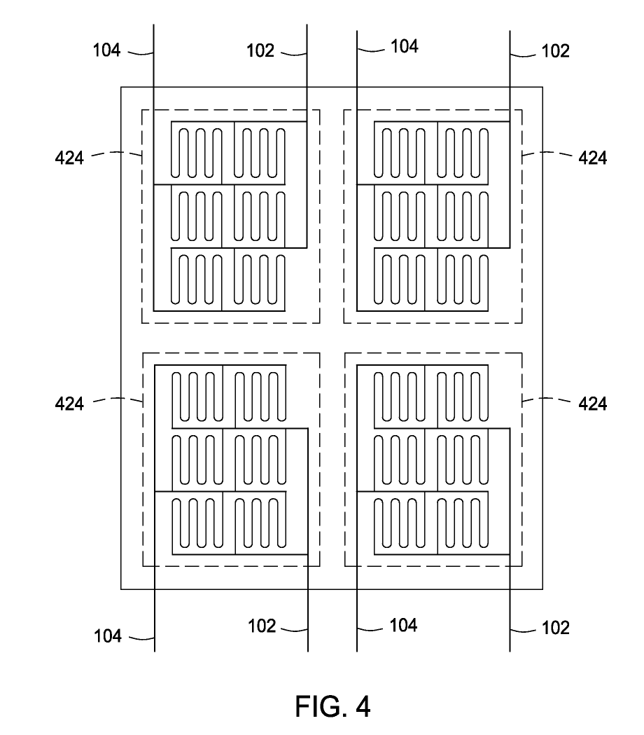

The heater layer 220 is configured to generate heat that is to be transferred to the patient to which the device is applied. In some embodiments, the heater layer 220 may include one or more heating elements, such as resistive heaters, disposed on a dielectric substrate 222. For example, as depicted in FIG. 2B (which illustratively depicts the thermal insulator 210 in phantom) an electrical circuit, or heater cell 224 may be provided to generate heat by Joule heating. Although a single heater cell 224 is shown, a plurality of heater cells may be provided (see, for example, FIG. 4). For example, a patient warming device may include tens, or hundreds, or thousands of heater cells. In addition, any one or all of the heater cells provided in a particular patient warming device may include one or more heating elements. For example, any one or more of the heater cells may include tens, or hundreds, or thousands of heater elements. The number and/or geometry of the heater cells and/or of the heater elements or other components of the heater cells may be varied as desired to provide, amongst other things, a desired power density per unit area, a desired granularity of control over the heaters, or the like. In some embodiments, each heater element may be about 0.50″ (1.27 cm) or less, or about 0.25″ (0.64 cm) or less, per side (e.g., the heater element may have a major cross-sectional area of about 0.25 in² (0.64 cm²) or less or about 0.06 in² (0.16 cm²) or less). Other dimensions of the heater elements, greater or smaller, may be used as well.

The dielectric substrate 222 is generally a flexible, polymer film, such as for example, polyethylene, polyester, or the like. In some embodiments, the heater cell may be formed by screen-printing conductive inks (such as polymer thick film inks or pastes) on the dielectric substrate 222. Wires or other electrically resistive materials are also expected to provide sufficient heat generation upon the application of current through the circuit and may also be used. In some embodiments, a protective dielectric coating or layer (not shown) may be disposed atop the heater cell or cells (or the heater elements of the cells) to protect the heater cells.

Each heater cell 224 may be coupled to an electrical power source (such as a battery, a power supply, a wall outlet, or the like) via leads 102, 104. Optionally, the leads 102, 104 may be terminated at a connector 250 that may be plugged into a mating connector coupled to the power supply for the patient warming device. For example, in some embodiments, where the patient warming device is configured to run on standard power provided, for example, via a wall outlet, the connector 250 may be a standard plug configured to interface with the wall outlet. Alternatively, in some embodiments, the connector 250 may be configured to plug, either directly or via a mating connector, into a battery pack, a transformer, a power supply, or the like. In embodiments were a plurality of heater cells are provided, the leads for providing power to each heater cell may be aggregated and terminated at a common connector for ease of coupling the heater cells to the source of power.

The heater layer may have various configurations. In general, the heater layer includes one or more heater cells disposed thereon. The heater cell or cells include one or more heater elements disposed thereon. Each heater element (or alternatively, groups of heater elements) may be independently controllable to provide the desired granularity of local heat control. The one or more heater elements may be provided in an electrical circuit that is connected to a power source. For example, in some embodiments, a plurality of independently controllable heater elements may be arranged in parallel with power supplied from a constant voltage source. In some embodiments, a plurality of independently controllable heater elements may be arranged in series with power supplied from a constant current source. In some embodiments where the heater elements are arranged in parallel with electrical power supplied from a constant voltage source, an independently controllable heater element could be a strip of positive temperature coefficient (PTC) resistive ink, or a resistive ink arranged in series with a PTC resistive ink, or a resistive ink arranged in series with an active electrical element that selectively switches off above a defined switching temperature. In some embodiments where the heater elements are arranged in series with electrical power supplied from a constant current source, an independently controllable heater element could be a strip of negative temperature coefficient (NTC) resistive ink, or a resistive ink arranged in parallel with an active electrical element (or configuration of elements) that serves to selectively shunt current through the element and away from the resistive ink when the independently controllable heater element exceeds some predefined switching temperature.

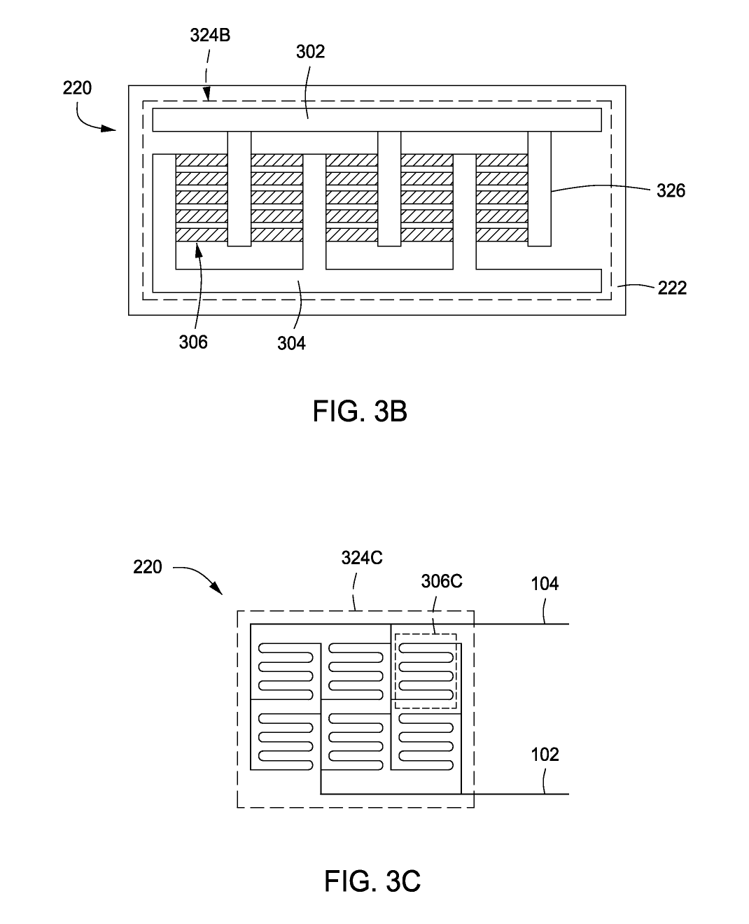

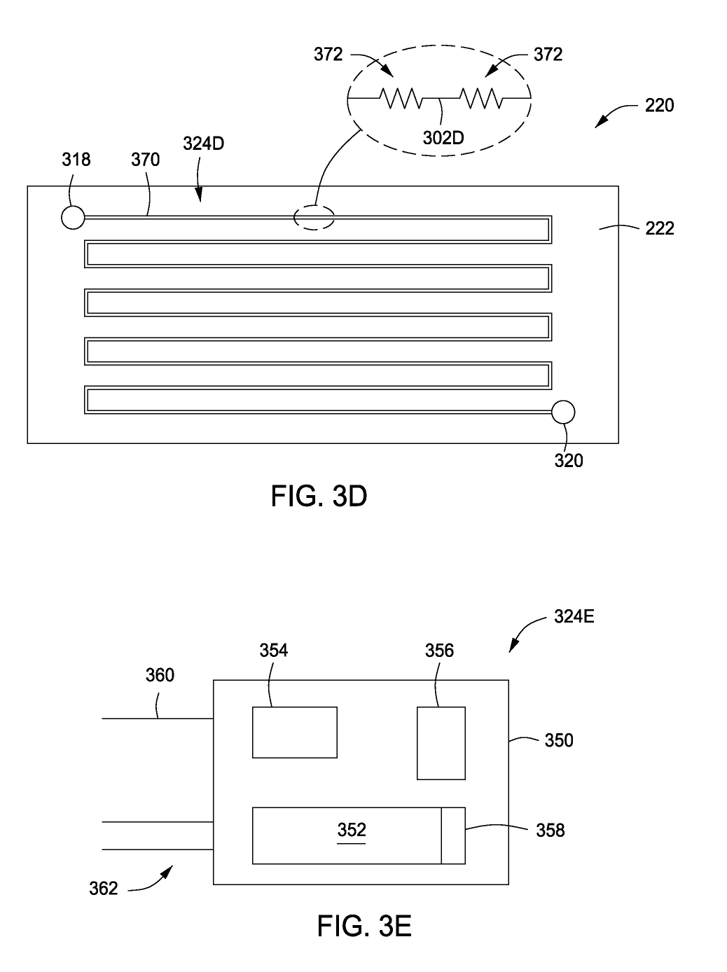

For example, FIG. 3A shows an illustrative top view of heater layer 220 having a heater cell 324A disposed thereon in accordance with some embodiments of the invention. The heater cell 324A is configured as a resistive heater that includes electrically conductive leads 302 and 304 having one or more electrically resistive paths 306 that bridge the electrically conductive leads 302 and 304. In some embodiments, the heater elements of the heater cell predominantly include the electrically resistive paths 306. The number and size of the electrically resistive paths 306 may be selected to control the surface density of the heater cell 324A. In some embodiments, the electrically resistive paths 306 may have a length approximately equal to or larger than the total thickness of the dielectric substrate 222 and the heat conduction layer 230. In some embodiments, the electrically resistive paths 306 may have a length that is greater than about 0.5 mm, or greater than about 1 mm, and up to about 15 mm. The leads, and other conductive portions of the heater cell 324A may comprise suitable conductive materials, such as discussed above. In some embodiments, a conductive ink, such as a silver-based electrically conductive paste, may be screen-printed or otherwise deposited and cured on the dielectric substrate 222 to form the electrically conductive leads 302, 304. The electrically resistive paths 306 may comprise the same or different materials than the leads 302, 304. In some embodiments, the electrically resistive paths 306 may comprise a carbon-based material. In some embodiments, the electrically resistive paths 306 may comprise a positive temperature coefficient material. Suitable examples of a screen-printable carbon-based ink include 7282, 7102, and 7105 Carbon Conductor inks available from DuPont Microcircuit Materials, of Research Triangle Park, N.C. Suitable examples of a screen-printable silver-based ink include 5000, 5021, 5025, and 5028 Silver Conductor inks also available from DuPont Microcircuit Materials.

In some embodiments, at least some of the electrically resistive paths 306, may be fabricated from a positive temperature coefficient (PTC) material, in which the electrical resistance increases, and in some embodiments, abruptly increases as the temperature approaches a switching temperature. In embodiments where the electrically resistive paths 306 comprise PTC materials, each electrically resistive path 306 may define the smallest independently controllable heater element of the patient warming device. In some embodiments, there is a benefit to arraying miniature PTC heaters across the heater layer; the use of miniature PTC resistive heaters minimizes hot spots across the patient warming device because each heater self-regulates independently. In some embodiments, the PTC material may be deposited on the substrate in a similar manner as discussed above. One source of a screen-printable positive temperature coefficient ink is 7282 PTC Carbon Resistor available from DuPont Microcircuit Materials. Other components of the heater cell, or portions thereof, may also be formed of PTC materials, alternatively or in combination with the electrically resistive paths 306. The switching temperature can be selected, depending on the design of the patient warming device, or the operation of a controller, according to a number of options. In one option, the switching temperature is at the temperature desired to be applied to the patient through the gel layer. In another option, the switching temperature is a temperature somewhat higher than the desired application temperature, and results in the desired application temperature at the body contact surface (due to the thermal impedance of the gel layer). In another option, the switching temperature is a temperature somewhat higher than the desired application temperature, which provides a safety backup for another temperature control mechanism. The desired application temperature (at the upper surface of the patient warming device) will typically be about 37 to 43° C. For humans, for example, a desired application temperature can be about 37 to 40° C. For an animal with a higher body temperature, it can be about 40 to 43° C.

The “switching temperature” for the purposes of this patent application shall be defined by the application temperature (provided to the patient). In some embodiments, the heating elements may supply power up to 1 watt per square inch. In some embodiments, there may be at least one heater element (or heater) per square centimeter. The heaters themselves may be substantially smaller than the area of the gel to which they supply heat. Where feasible, for the purpose of calculating such density, the area taken by a heating element is, in part, measured by the median boundaries between the heating elements. For the boundaries to the peripheries (thus not measured by the method of the above sentence), the area is measured by the lines most symmetrical to the lines defined by the interior boundaries. Hence, where this method is feasible, the density is the total number of heating elements over the sum of the areas so defined. Where this area measuring method is not feasible, the guiding principles are to find symmetry where possible, and to avoid counting excessive area to the peripheries that is not involved in heating.

In operation, in some embodiments, a parallel arrangement of PTC resistive heaters (e.g., electrically conductive paths 306) may be coupled via leads 102, 104 to a power source that provides a voltage differential (e.g., a voltage source). In some embodiments, the voltage source may be a constant voltage source. As the PTC resistive heaters generate heat and increase in temperature, the resistance of the PTC resistive heaters increases. Based upon the selection and configuration of the PTC resistive heaters, as the temperature approaches a predefined limit, the increased resistance will cause a shift in the current flow to other PTC resistive heaters, or other current flow paths provided in the parallel arrangement. The reduction in current flow will cause the PTC resistive heater to cool, lowering its resistance, which in turn, will increase the current flow therethrough. Such a configuration allows for the self-regulation, or independent control, over each individual PTC resistive heater.

The heater cells can be prefabricated and coupled to the heater layer substrate, or formed directly on the heater layer substrate, for example, by screen printing methods, or other printing methods for forming electrical traces, such as utilizing ink annealed with heat or solvent evaporation. PTC materials are typically formed (a) of polymer particles and conductor particles (e.g., polymer-based PTC materials), such that—it is believed—volume increases at the glass transition temperature cause conductor particles to separate, or (b) with certain ceramic materials (e.g., ceramic-based PTC materials), with the resistance increase resulting—it is believed—from grain boundary effects. Inks can contain particles of a crystalline polymer (such as HD polyethylene, or the like), and particles of a conductor (such as graphite, silver, or the like). The choice of polymer particles, conductor particles, the ratios therebetween, and the like can be varied to change the temperature at which the heaters lose heat output (e.g., the switching temperature). Ceramic PTC materials are often titanate ceramics. The amount of PTC material should be sufficient to provide the necessary drop in resistance that provides the switching temperature.

The heater cell 324 (and any other embodiments of the heater cell disclosed herein) may be coupled to a power source, for example, by leads (leads 102, 104 shown in FIGS. 2A-B) coupled to the electrically conductive leads 302 and 304. For example, as shown in FIG. 3A, contact portions 318 and 320 may be provided on the conductive leads 302, 304 to facilitate coupling to respective leads (not shown) that may be further coupled to a power source (as depicted in FIG. 5). At least the contact portions 318, 320 of the leads 302, 304 may comprise a material suitable for reliably coupling to the power source. The leads from the power source may be coupled to the contact portions 318 and 320 in any suitable manner, such as by soldering, brazing, bonding with conductive adhesives, clamping, or the like, such that a robust electrical and mechanical coupling is provided. Upon the application of a voltage between 302 and 304, current flows through the electrically resistive paths, thereby generating heat which is transmitted through the dielectric substrate 222 (and thermal conduction layer 230, when present) to the skin of the patient.

In some embodiments, one or more sensors may be provided to sense the temperature (or a metric corresponding to temperature) of the patient warming device as a whole, or to portions of the patient warming device (such as individual heater cells or groups of heater cells). The sensor may be coupled to a controller (e.g., controller 502, discussed below) that is also coupled to a power supply or power regulator (e.g., power supply 504, discussed below) to verify proper functioning of the patient warming device 100 and/or to provide closed loop control over the temperature of the heating elements on the heater layer 120. For example, the sensed metric may be fed back to the controller controlling the patient warming device to facilitate more precise control over the temperature of the patient warming device. For example, the sensed temperature feedback may be used to turn off the entire patient warming device or sections of the patient warming device, or the feedback may be used to control the power supplied to one, some, or all of the patient warming device heater cells. In some embodiments, and as depicted in FIG. 3A, a sensor 310 may be provided proximate the heater cell 324A.

The sensor 310 may include any suitable sensor for measuring the temperature of the patient warming device, portions thereof, and/or the patient. For example, the sensor 310 may be used to measure the temperature of heater elements, the temperature of portions of the patient warming device other than the heater elements, a patient's skin temperature, or the like. In some embodiments, the sensor 310 may comprise electrically conductive pads 312, 314, that are bridged by a thermal sensor 316. In some embodiments, the thermal sensor 316 comprises a conductive material having a well-characterized temperature-resistance relationship that can be utilized by the controller to determine the temperature of the thermal sensor 316 by measuring the resistance of the thermal sensor 316. In some embodiments, the thermal sensor 316 comprises a positive temperature coefficient material, such as a PTC printed thermistor. In operation, as the temperature of the heater cell 324A approaches a defined switching temperature (i.e., the switching temperature of the positive temperature coefficient material), a large increase in electrical resistance of the thermal sensor 316 is sensed, thereby allowing for a closed-loop control circuit to decrease the voltage across leads 302, 304, which, in turns, decreases the amount of heat generated by the heater cell 324A. Alternatively or in combination, a prefabricated sensor may be disposed proximate the heater cell to monitor the temperature of the patient warming device near the heater cell.

Alternatively or in combination, the resistance across the whole heater cell could be sensed (i.e., the heater cell itself acts as the sensor). For example, FIG. 4 shows a collection of four heater cells 424 that operate as a single cell when the individual leads 104 are connected and the individual leads 102 are connected. When electrical communication between the individual leads 104 is broken (and similarly for the individual leads 102), the resistance across the four heater cells 424 can be measured independently and power can be supplied to each heater cell independently. In this way, the heater cells can be used in combination with a closed-loop control circuit to provide independent temperature control across the heater surface of the patient warming device, where the area of independent temperature control is similar to the area of the heater cell. In some embodiments, switches may be provided to control each heater cell via the controller based upon the sensed resistance of the heater cell.

Although depicted as being adjacent to the heater cell, the sensor may be disposed overlying or underlying the heater cell or in other suitable locations for sensing the temperature as desired. For example, in some embodiments, the sensor may alternatively or in combination with other sensors, be disposed over or more closely adjacent to the heater cell (or heater elements of the cell) to more accurately measure the temperature of the heater cell, or of one or more heater elements within the heater cell. A plurality of sensors may also be provided, disposed proximate to some or all of the heater cells in a patient warming device or wherever desired to control the operation of the patient warming device. In some embodiments, a thermal sensor, external to the patient warming device, may be placed in thermal communication with a heater element or heater cell of the patient warming device to allow for external calibration of the heater element or heater cell so that the heater cell itself may be used for accurate thermal sensing as described above. The external thermal sensor may be an accurate NTC thermistor, thermocouple, or optical sensor, that is separately provided or that is part of the connector and cable that controls power delivered to the heater cell. A calibration method could consist of placing the external thermal sensor in communication with a heater cell and without the power delivered to the patient warming device simultaneously measuring the temperature of the heater cell with the external thermal sensor and the electrical resistance of the heater cell to establish a single point temperature calibration of the heater cell as a temperature sensor itself. A multi-point calibration curve may be constructed by applying power to the heater cell and simultaneously measuring and pairing the heater cell temperature and resistance. This type of calibration approach may be a useful way of removing part to part differences between different patient warming devices that may arise in manufacturing, especially when the heater cells are screen printed with polymer thick films and inadequate control over the film thickness (and consequently electrical resistance) does not allow for the heater cells to be used as thermal sensors without calibration.

Alternatively or in combination, in some embodiments, the sensor may be at least partially thermally insulated from the heater elements so that the sensor may more accurately measure the temperature of a patient through the patient warming device, rather than the temperature of the heater elements themselves. In such embodiments, a layer 322 of thermally insulative material may be provided between the sensor 310 and the resistive heaters (e.g., the electrically conductive paths 306). In some embodiments, the layer 322 may surround, or substantially surround, the sensor 310 or may be disposed between the sensor 310 and the resistive heaters of the heater cell. In some embodiments, the sensor 310 may be disposed beneath the heater layer and the layer 322 disposed between the sensor 310 and the heater layer.

As noted above the configuration and selection of materials comprising each heater cell may be selected as desired to provide heater cells of a desired size and/or geometry. For example, FIG. 3B is a plan view of a heater cell 324B in accordance with some embodiments of the present invention. In the embodiment depicted in FIG. 3B, a plurality of positive temperature coefficient bridges 326 are coupled between the lead 302 and the electrically resistive paths 306 that couple the lead 302 to lead 304. As described above, when the heater cell approaches a defined switching temperature, a large increase in the resistance of the positive temperature coefficient bridges 326 limits the current carried through the circuit which, in turn, limits the heat generated by the circuit. Alternatively, the entire lead 302 may comprise a positive temperature coefficient material that when heated above a defined switching temperature displays a large rise in resistance, thereby decreasing the current carried through the circuit and limiting the heat generated by the circuit.

In some embodiments, as depicted in FIG. 3C, the plurality of electrically resistive paths 306 may have a different number or geometry than that shown in FIGS. 3A and 3B. It is contemplated that other geometries may also be used in accordance with the teachings disclosed herein.

In some embodiments, some or all of the heater cells of the heater layer may comprise negative temperature coefficient (NTC) resistors, or heater elements, arranged in series, and driven with a current source, for controlled delivery of heat to the patient. In some embodiments, the current source may be a constant current source. Because each NTC heater element self-regulates independently, miniature NTC resistive heaters may be used to minimize hot spots across the patient warming device. For example, FIG. 3D shows an illustrative top view of the heater layer 220 having a heater cell 324D in accordance with some embodiments of the invention. The heater cell 324D may be substantially similar in composition, configuration, and operation, as the heater cell 324A discussed above, except as discussed below. As depicted in FIG. 3D, in some embodiments, the heater layer 220 may include a heater cell 324D disposed on the substrate 222. The heater cell 324D may include a conductive path 370 disposed between contact portions 318, 320. The conductive path 370 is formed by series-connected resistive elements 372 disposed along an electrically conductive lead 302D (as shown in the detail of FIG. 3D). The resistive elements 372 may comprise any of the materials discussed above with respect to the electrically conductive paths 306, except that negative temperature coefficient (NTC) materials may be used instead of PTC materials.

The heater cell 324D may be several millimeters on a side (e.g., from about 1 to about 5 mm per side) or it may be the substantially the size of the heater layer 220. The size of the heater cell 324D may be determined by the magnitude of the resistance per unit length for the NTC heater elements and the magnitude of the available current. In some embodiments, a plurality of cells, for example, arrayed in an XY plane across a complete heater layer and all driven independently by a current source. As described above with PTC heater elements, the resistance across the whole NTC heater cell or heater element could be sensed thereby allowing the heater cell or element itself to act as the sensor and also allowing for the possibility of closed-loop control of the temperature of the NTC heater cell.

In the case of a negative temperature coefficient resistor arranged in series and driven with a current source, the width of the resistor trace defines the dimension over which independent control of temperature may be provided. Here, because the width is typically smaller than the length of the resistor trace between the electrically conductive leads (e.g., 318 and 320), the minor dimension in the plane of the patient warming device defines the dimension for independent temperature control. In the case of positive temperature coefficient resistors arranged in parallel and driven with a voltage source, the spacing between conductors (or the resistive bridge length) defines the dimension over which independent temperature control may be provided. Here, the minor dimension of a resistor trace (e.g., 306) in the plane of the patient warming device is typically the resistive bridge length and this minor dimension defines the dimension for independent temperature control. In some embodiments, where the heater element comprises PTC or NTC materials, the temperature, and thus the electrical characteristics, across a single heater element may vary, and thus a single heater element may behave similarly as described herein with respect to a plurality of heater elements.

Alternatively or in combination, in some embodiments, some or all of the heater cells of the heater layer may comprise microcircuits. For example, as depicted in FIG. 3E, a heater cell 324E is shown that includes a microcircuit 350. In some embodiments, the microcircuit 350 includes heater circuitry 352, control logic circuitry 354, a temperature sensor 356, switching circuitry 358, power leads 362 to facilitate coupling the microcircuit to a source of power (e.g., similar to leads 102, 104 discussed herein), and a control lead 360. The heater circuitry 352 comprises one or more resistive heaters for generating heat in response to a current flowing through the circuitry. The switching circuitry 358 may be provided to selectively couple the elements of the heater circuitry 352 to the power supply. The switching may be provided as an on/off switch controlling the entire heater circuitry 352, to single elements of the heater circuitry 352, to subsets of the elements of the heater circuitry 352, or to combinations of the above. The control lead 360 provides one or more leads for communicating to and/or from the microcircuit 350. The control lead 360 may facilitate coupling the microcircuit 350 to other components for receiving or transmitting data to/from the microcircuit 350. For example, the control lead 360 may be coupled to another controller remote from the microcircuit 350 (as discussed below) for remote control or collection of data, a source of input data (such as from a remote temperature sensor), to a display, to an alarm, or the like.

In some embodiments, the control logic circuitry 354 may be provided to control the operation of the microcircuit 350. For example, the control logic circuitry 354 may control the operation of switching circuitry 358 that may open or close switches coupled between one or more elements of the heater circuitry 352 to increase or decrease the heat load generated by the heater circuitry 352. In some embodiments, such control may be provided in response to data provided by the temperature sensor 356. As discussed above, the temperature sensor 356 may be separate circuitry provided in the microcircuit 350 or may be part of other circuitry (such as the heater circuitry 352). Alternatively, in embodiments were no temperature sensor 356 is present, such control may be provided in response to data provided by a temperature sensor disposed elsewhere in the patient warming device.

In some embodiments, the control of the microcircuit 350 (or a plurality of microcircuits 350) may be provided remotely, for example, via a controller in communication with the patient warming device via the control lead 360 (such as the controller 502 discussed below with respect to FIG. 5). In such embodiments, the control logic circuitry 354 may be omitted from the microcircuit 350.

The number of heating elements (or heater cells) may depend upon the overall size of the patient warming device 100. The number of heating elements may also depend upon a desired size of each heating element. It can be desirable to have as many heating elements as practical so that each can each respond to the heat load at the corresponding points of contact. The overall size of the patient warming device should be such as to supply the heat energy necessary to counteract the effects of heat convection from a patient laying exposed, for example, on an operating table, without exceeding maximum desired patient surface temperatures. The use of separate heating elements or heater cells, each with an upper temperature limit, helps minimize the formation of hot spots, for example hot spots at locations on the patient heating device where heat is not being efficiently conveyed to the patient, such as where a heating device has separated from the patient. In addition, smaller heater cells may reduce the number of potential hot spots in the patient warming device 100. In some embodiments, the interdigitated conductive paths 318 and 320 are closely spaced, with a typical range between 0.5 mm and 15 mm, so that the individual heating elements 306 are short, thereby defining the minimum lateral dimension that can be independently controlled for heat generation. Alternatively or in combination, hot spots can also be limited by selection of the gel thickness, as discussed above.

In some embodiments, the heater cells may be coupled together in parallel, such that the effect of disconnection, or failure, of one or more of the heater cells may have little or no impact on the performance of the patient warming device. In such embodiments, the patient warming device may also be customizable in size to fit a particular application. For example, portions of the patient warming device may be cut off to make the patient warming device smaller to fit on a smaller patient, or to provide additional access to a location on the patient proximate the desired location of the patient warming device. Cut lines or other demarcations may be provided to indicate locations where it is safe to cut the patient warming device without cutting through essential components of the device (such as the leads to the power source).

FIG. 5 shows a patient warming device 100 with the layers shown separated for illustrative purposes, coupled to a power supply 504 (or power regulator) and, optionally, to a controller 502. The power supply 504 may be any suitable source or supply of power to operate the patient warming device, such as a constant voltage source, a constant current source, an DC power supply, an AC power supply, facilities power (such as a wall outlet), or the like. A sensor 506 (similar to sensor 310) may be provided and coupled to the controller 502 to verify proper functioning of the patient warming device 100 and/or to provide closed loop control over the temperature of the heating elements on the heater layer 120, in the manner as discussed above.

The controller 502 generally comprises a central processing unit (CPU) 510, a memory 512, and support circuits 514 for the CPU 510 and may be coupled to and may control the heater cells (individually or grouped) of the heater layer 220, and/or is coupled to and controls the power supply 504 for the heater layer 220, and is coupled to data inputs comprising data from the sensor(s) 506 or from the heater cells 224 (such as the current consumption of the heaters), and or data from a temperature monitor (not shown) coupled to a patient being warmed by the patient warming device 100. The controller 502 may be one of any form of general-purpose computer processor that can be used in an industrial or medical setting for controlling various devices and sub-processors. The memory, or computer-readable medium, 512 of the CPU 510 may be one or more of readily available memory such as random access memory (RAM), read only memory (ROM), flash memory, floppy disk, hard disk, or any other form of digital storage, local or remote. The support circuits 514 are coupled to the CPU 510 for supporting the processor in a conventional manner. These circuits can include cache, power supplies, clock circuits, input/output circuitry and subsystems, and the like. Operational protocols for the patient warming device may be stored in the memory 512 as a software routine that may be executed or invoked to control the operation of the patient warming device in the manner described herein. The software routine may also be stored and/or executed by a second CPU (not shown) that is remotely located from the hardware being controlled by the CPU 510.

The controller 502 can be responsive to inputs designating whether the patient warming device is to be used to warm the entire patient, with the localized heat conveyed through the patient's circulatory system, or to predominately warm a local area. Local area heat can be useful in physical therapy, injury treatments, or the like.

As indicated above, the patient warming device can be operated with no external controller, instead utilizing just its intrinsic heat control features of the a heater layer comprising PTC heater cells or microcircuits and, optionally, temperature sensors. As such, the patient warming device may be utilized to reduce heat spikes that could be generated in the individual heater cells without actively controlling each heater cell. Such a configuration facilitates controlling the heat energy provided to a patient rather than the temperature of the device. In other embodiments, the patient warming device can be operated with minimal control elements. These options facilitate use in a highly transportable form, such as operated by a battery pack and/or free of a complex console.

The materials of the patient warming device are generally selected so that the device overall is sufficiently flexible to conform to a patient. As mentioned above, in some embodiments the thermal conduction layer may be disposable. It is also anticipated that the entire patient warming device can be disposable, since it is believed that it can be made in price range that makes disposable use practical. Since the patient warming device operates without air or water flow, it is simpler to operate, does not require air and/or water flow control mechanisms, and does not provide fluid flows that create a contamination risk in a sterile area. In addition, although described above in terms of certain layers and materials, it is contemplated that variations of the above embodiments can be made in keeping with the scope of the present invention. For example, different materials may be utilized that are compatible with or provide similar functions as described above, or additional layers may be provided that do not substantially interfere with the operation of the patient warming device.

While the foregoing is directed to embodiments of the present invention, other embodiments of the invention may be devised without departing from the basic scope thereof. Any claim below that is written as dependent on an independent claim can also be written as dependent on any of the claims under such independent claim, except where logic forecloses such a dependency.

What is claimed is:

1. A patient warming device, comprising:
a heater layer comprising a plurality of heater cells disposed on a flexible substrate; and
a thermal conduction layer disposed on a patient side of the heater layer to transfer heat generated by the heater layer to a patient.

2. The patient warming device of claim 1, wherein each heater cell comprises an electrical circuit to generate heat by Joule heating.

3. The patient warming device of claim 2, wherein one or more of the heater cells comprises one or more heater elements.

4. The patient warming device of claim 3, wherein at least some of the heater elements are at least partially formed of a positive temperature coefficient material configured to reduce the current flowing through the resistive heater upon approaching a switching temperature.

5. The patient warming device of claim 4, wherein the switching temperature is selected to provide an application temperature of about 37 to about 43° C.

6. The patient warming device of claim 4, wherein the positive temperature coefficient material is a polymer-based positive temperature coefficient material

7. The patient warming device of claim 4, wherein the heater elements are electrically arranged in parallel and coupled to a voltage source.

8. The patient warming device of claim 3, wherein at least some of the heater elements are at least partially formed of a negative temperature coefficient material configured to reduce the resistance the resistive heater upon approaching a switching temperature.

9. The patient warming device of claim 8, wherein the switching temperature is selected to provide an application temperature of about 37 to about 43° C.

10. The patient warming device of claim 8, wherein the heater elements are electrically arranged in series and coupled to a current source.

11. The patient warming device of claim 3, wherein the variation of electrical resistance with temperature of a first heater cell is used to sense the first heater cell temperature.

12. The patient warming device of claim 3, wherein one or more of the heater cells comprises a microcircuit.

13. The patient warming device of claim 12, wherein the microcircuit comprises heater circuitry.

14. The patient warming device of claim 13, wherein the microcircuit further comprises a control logic circuitry to control the heater circuitry.

15. The patient warming device of claim 14, wherein the microcircuit further comprises switching circuitry coupled to the heater circuitry, and wherein the control logic controls operation of the switching circuitry to selectively enable or disable heater elements of the heater circuitry.

16. The patient warming device of claim 14, wherein the microcircuit further comprises a sensor configured to provide a metric correlating to temperature to the control logic.

17. The patient warming device of claim 12, wherein the microcircuit further comprises a sensor configured to provide a metric correlating to temperature to a microcircuit controller disposed remote from the microcircuit.

18. The patient warming device of claim 12, wherein a plurality of the heater cells comprise microcircuits and further comprising a microcircuit controller disposed remote from the microcircuit and coupled to each of the plurality of microcircuits.

19. The patient warming device of claim 1, wherein the thermal conduction layer comprises a hydrogel.

20. The patient warming device of claim 1, wherein the thermal conduction layer is thermally coupled to the heater layer.

21. The patient warming device of claim 1, wherein the thermal conduction layer is fabricated from a material that lowers the thermal impedance of skin upon contact therewith.

22. The patient warming device of claim 1, wherein the thermal conduction layer is flexible and conformable so as to remove an air barrier between the thermal conduction layer and a patient's skin during use of the patient warming device.

23. The patient warming device of claim 1, wherein the thermal conduction layer conforms to the pores of a patient's skin during use of the patient warming device.

24. The patient warming device of claim 1, wherein the thermal conduction layer is disposed directly in contact with the heater layer.

25. The patient warming device of claim 1, wherein the heater cells are wired in parallel and arranged to allow a portion of the patient warming device to be removed without impairing the operation of the remaining portion of the patient warming device.

26. The patient warming device of claim 25, further comprising visible demarcations provided to show where the patient warming device may be cut without impairing the operation of the patient warming device.

27. The patient warming device of claim 1, wherein the patient warming device is flexible to facilitate conforming the patient warming device to a patient during use.

28. The patient warming device of claim 1, further comprising:
a controller to control the operation of the heater cells.

29. The patient warming device of claim 28, further comprising:
a sensor configured to provide a metric correlating to the temperature of a location of the patient warming device to the controller.

30. The patient warming device of claim 29, wherein a plurality of sensors are provided corresponding to each of the plurality of heater cells.

31. The patient warming device of claim 29, wherein the sensor comprises a PTC printed thermistor.

32. The patient warming device of claim 29, wherein one or more of the heater cells comprise a heater element, wherein the sensor comprises the heater element, and further comprising a second sensor configured to provide a metric correlating to the temperature of the heater element.

33. The patient warming device of claim 1, further comprising:
a power supply coupled to the heater cells.

34. The patient warming device of claim 1, further comprising:
a thermal insulator disposed on a non-patient side of the heater layer.

35. A method of elevating the temperature of a patient or a portion of a patient, comprising:
applying the patient warming device of claim 1 to the patient; and
generating heat with the patient warming device so that heat is conveyed to the patient.

36. A patient warming device kit, comprising:
a patient warming device setup including a heater layer comprising a plurality of heater cells; and
a thermal conduction layer adapted to be removably disposed on the patient warming device setup to form a patent warming device, wherein the thermal conduction layer is adapted to be disposed between the patient and the heater layer, wherein the patient warming device is adapted to be flexible to facilitate conforming to the patient.

37. The patient warming device kit of claim 36, wherein the patient warming device setup further comprises:
a thermal insulator disposed on the non-patient side of the heater layer.

38. The patient warming device kit of claim 36, wherein the patient warming device kit is packaged in a pack that is suitable for sterilization.

39. A method of elevating the temperature of two or more patients or portions of the patients, comprising:
applying the patient warming device of claim 1 to a first patient;
generating heat with the patient warming device so that heat is conveyed to the first patient;
replacing the thermal conduction layer of the patient warming device;
applying the patient warming device to a second patient; and
generating heat with the patient warming device so that heat is conveyed to the second patient.
